(19) United States
(12) Patent Application Publication
(10) Pub. No.: US 2023/0210519 A1
(43) Pub. Date: Jul. 6, 2023

(54) JOINTED DEVICE FOR AUTOMATIC SUTURE AND METHOD FOR SAME

(71) Applicant: Health Research, Inc., Buffalo, NY (US)

(72) Inventor: Khurshid GURU, East Amherst, NY (US)

(21) Appl. No.: 18/001,001

(22) PCT Filed: Jun. 7, 2021
(86) PCT No.: PCT/US2021/036271

§ 371 Date: Dec. 7, 2022

Related U.S. Application Data
(60) Provisional application No. 63/035,820, filed on Jun. 7, 2020.

(51) Int. Cl. A61B 17/04

(52) U.S. Cl. CPC A61B 17/0469; A61B 17/0467; A61B 2017/06052

(57) ABSTRACT

A device for automatic suturing includes a shaft having a longitudinal axis. The shaft has a joint rotatable about a joint axis which is at an angle>0° to the longitudinal axis. The device includes a clamp at a distal end of the shaft. The clamp is configured to be positioned over a length of tissue to be sutured. A driver is configured to engage with a screw of the shaft so that the driver translates along a length of the shaft when the driver is rotated. A helical needle is connected to the driver such that rotation of the driver causes rotation of the helical needle. In this way, the needle is advanced or retracted over a length of the clamp and over the joint of the shaft.

### CROSS-REFERENCE TO RELATED APPLICATIONS

This application claims priority to U.S. Provisional Application No. 63/035,820, filed on Jun. 7, 2020, now pending, the disclosure of which is incorporated herein by reference.

### FIELD OF THE DISCLOSURE

The present disclosure relates to suturing, and in particular for suturing during a minimally-invasive procedure.

### BRIEF SUMMARY OF THE DISCLOSURE

In a first aspect, the present disclosure may be embodied as a device for automatic suturing. The device includes a shaft having a longitudinal axis. The shaft has a joint rotatable about a joint axis which is at an angle>0° to the longitudinal axis. The device includes a clamp at a distal end of the shaft. The clamp is configured to be positioned over a length of tissue to be sutured. A driver is configured to engage with a screw of the shaft so that the driver translates along a length of the shaft when the driver is rotated. A helical needle is connected to the driver such that rotation of the driver causes rotation of the helical needle. In this way, the needle is advanced or retracted over a length of the clamp and over the joint of the shaft. In some embodiments, the helical needle further comprises one or more windows. In some embodiments, a tip of the helical needle is at a radially exterior portion of the helical needle for movement over the joint of the shaft. The helical needle may be configured to be advanced over an exterior of the clamp.

In some embodiments, the device further comprises a suturing thread having a leading end near a tip of the helical needle. The helical needle may be hollow and the suturing thread is disposed through the helical needle. The device may further comprise at least one cutter for severing the suturing thread. The suturing thread has a diameter, the helical needle has an inside diameter, and the ratio of the suturing thread diameter to the helical needle inside diameter is in the range of 1:6-1:10, inclusive. In some embodiments, the ratio of the suturing thread diameter to the helical needle inside diameter is greater than 1:8.

The device may further have a lock for reversibly locking the clamp in a closed position. The device may further have a shaft attached to the clamp, and the helical needle may be configured to be disposed over the shaft when in a retracted position. In some embodiments, a motor is operably connected to the driver.

In another aspect, the present disclosure may be embodied as a method for automatic suturing. The method includes engaging a clamp of a device according to any one of the disclosed embodiments over a first portion of tissue to be sutured. The clamp may be locked in place (e.g., locked in a closed position). The helical needle of the device is advanced along a length of the clamp such that the helical needle passes through the tissue to be sutured. A leading end of a suturing thread is attached near a tip of the helical needle. In this way, the suturing thread is pulled through the tissue with the helical needle. The method includes capturing the leading end of the suturing thread and retracting the helical needle back through the tissue so as to leave the suturing thread in place. The clamp may then be disengaged from the tissue.

The leading end of the suturing thread and/or a trailing end of the suturing thread may be fixed to form one or more sutures. For example, the leading end of the suturing thread and/or the trailing end of the suturing thread may be fixed by knotting.

The needle may be advanced using, for example, a motor (e.g., an electric motor). The helical needle may be advanced continuously, or selectively (e.g., in single rotation increments).

provides a method for automatic suturing. The method includes engaging a clamp over a length of tissue to be sutured. In some embodiments, the clamp may be locked in place over the tissue (e.g., over the length of tissue). A helical needle is advanced along a length of the clamp such that the helical needle passes through the tissue to be sutured. A leading end of a suturing thread may be attached at a tip of the helical needle. The leading end of the suturing thread is captured, for example, when the tip of the helical needle is advanced to an end position.

In some embodiments, a removable tip of the needle is captured with the leading end of the suturing thread. In such embodiments, the helical needle is retracted without the tip. The helical needle is retracted back through the tissue so as to leave the thread in place. The method includes disengaging the clamp from the tissue.

The leading end of the thread may be fixed to form one or more sutures. Similarly, a trailing end of the suturing thread may be fixed to form one or more suture. In some embodiments, both the leading end and the trailing end are fixed to form one or more suture. For example, sutures may be formed at each of the leading end and the trailing end. The leading end of the suturing thread and/or the trailing end of the suturing thread may be fixed using knots (e.g., by knotting the ends).

In some embodiments, the method may include cutting the leading end of the suturing thread so as to separate the tip of the helical needle from a thread portion to remain in place following retraction of the helical needle (e.g., a thread portion forming a suture).

The helical needle may be advanced and/or retracted by an electric motor. The helical needle may be advanced in single rotation increments. The helical needle may be advanced in increments less than a full rotation. The helical needle may be advanced in increments greater than a full rotation. The helical needle may be advanced to the end position.

In another aspect, the present disclosure may be embodied as a device for automatic suturing. The device comprises a clamp. The clamp is configured to be positioned over a length of tissues to be sutured. A lock may be provided for reversibly locking the clamp in a closed position. A helical needle is configured to be advanced and retracted along a length of the clamp. For example, the helical needle may be advanced along an interior portion of the clamp. In another example, the helical needle may be advanced over an exterior portion of the clamp. The needle has a tip, and a suturing thread having a leading end is attached to the tip of the helical needle. The needle may be hollow and the thread may be disposed through the needle.

The device may include a trap configured to capture a leading end of the thread once the helical needle has been advanced along at least a portion of the length of the clamp. The tip of the needle may be removable. The trap may be configured to capture the tip of the needle with the leading end of the thread.

The clamp may include a helical guide configured to guide the helical needle during advancement and retraction. The guide may be configured on an interior portion of the clamp. The device may further include at least one cutter for severing the suturing thread.

### DESCRIPTION OF THE DRAWINGS

For a fuller understanding of the nature and objects of the disclosure, reference should be made to the following detailed description taken in conjunction with the accompanying drawings, in which:

FIG. 1 is a diagram depicting a device according to an embodiment of the present disclosure;

FIG. 2A is a diagram of the device of FIG. 1, wherein the clamp is shown in an open configuration;

FIG. 2B is a diagram of the device of FIGS. 1-2A, wherein the clamp is shown closed;

FIG. 3A is a diagram showing a detail view of a clamp of the device of FIGS. 1-2B, wherein the clamp is shown in an open configuration;

FIG. 3B is a diagram showing a detail view of the clamp of the device of FIGS. 1-3A, wherein the clamp is shown closed;

FIG. 4 is a diagram of the device of FIGS. 1-3B, wherein a needle has been advanced over the clamp;

FIG. 5 is a diagram showing a detail of the clamp and needle of FIG. 4;

FIG. 6 shows a needle according to another embodiment of the present disclosure;

FIG. 7 shows a detail view of a tip of the needle of FIG. 6;

FIG. 8 is a chart showing a method according to another embodiment of the present disclosure;

FIG. 9 is a diagram of device according to another embodiment of the present disclosure;

FIG. 10A is a detail view of the driver and needle of the device of FIG. 9;

FIG. 10B is a partial exploded view of the driver and needle of FIG. 10A; and

FIG. 11 is a diagram depicting a driver head and needle according to another embodiment of the present disclosure.

### DETAILED DESCRIPTION OF THE DISCLOSURE

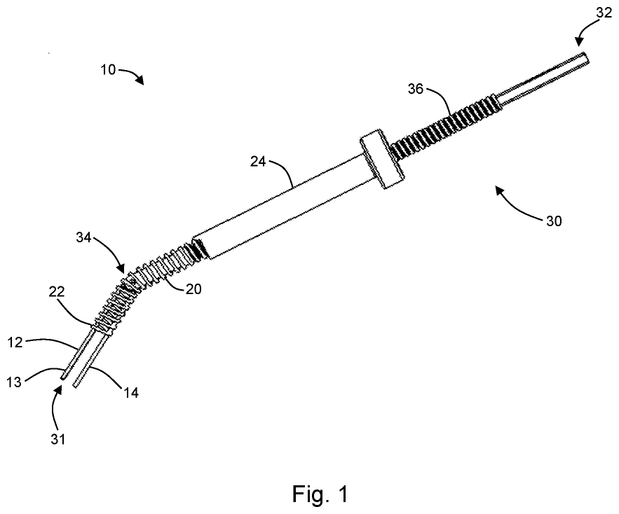

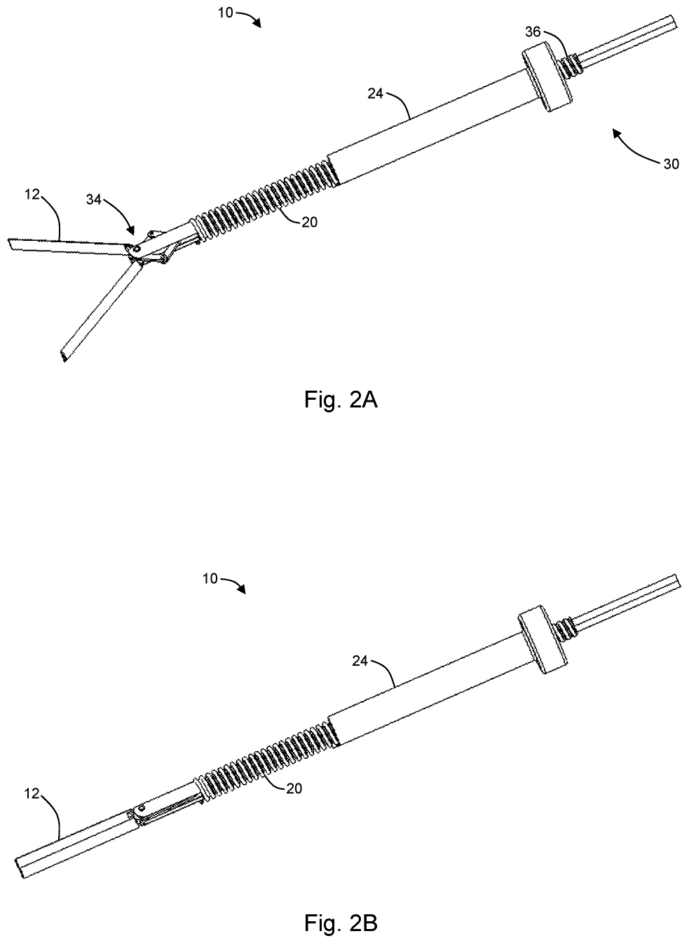

With reference to FIG. 1, in an aspect, the present disclosure may be embodied as a device 10 for automatic suturing. The device 10 includes a shaft 30 having a longitudinal axis. A clamp 12 is located at a distal end 31 of the shaft. In an exemplary embodiment, the clamp 12 includes a first side portion 13 and a second side portion 14, each hingedly connected to the other. A spring may cause the first and second side portions 13,14 to be biased toward each other—i.e., biased to a “closed” state of the clamp 12. Such a clamp 12 is configured to be opened and positioned over a length of tissue. The clamp 12 may be engaged by allowing the clamp to return to the closed position (e.g., partially closed as the tissue(s) may now cause some separation at an opening of the clamp 12). In other embodiments, the clamp is not spring biased and the clamp is operably moved from an open state to the closed state and varying degrees therebetween by a user.

In some embodiments, the device 10 includes a joint 34 at a location of the shaft 30 proximally-located with respect to the clamp 12. In this way, a distal portion of the shaft including at least the clamp is able to rotate into a position off of (i.e., at an angle>0° to) the longitudinal axis of the shaft. For example, the joint may have a rotational axis which is orthogonal to the longitudinal axis of the shaft. A joint control member is disposed through the shaft such that the joint is operable from a proximal end 32 of the shaft.

The device 10 further comprises a helical needle 20 movably disposed over a portion of the shaft 30 and/or the clamp 12. A driver 24 is connected to a proximal end 22 of the needle 20 such that rotation of the driver 24 about the longitudinal axis of the shaft will cause the needle 20 to rotate about the shaft. Similarly, translation of the driver 24 along the length of the shaft will cause the needle 20 to translate as well. The shaft 30 includes a screw 36 with which the driver 24 is engaged. In this way, rotation of the driver will cause the driver to advance along the screw (or withdrawn if rotated in an opposite manner) thereby also translating the driver (and needle) along the length of the shaft. The screw may have a trapezoidal thread form, (for example, an ACME thread profile) or another thread form. The driver may have a length such that it can be manually operated from outside of the body when in use. In some embodiments, the driver may be advanced using a motor, such as an electric motor. In such embodiments, the driver may have a shorter length such that the driver is completely contained within the body when in use, or a longer length such that at least a portion of the driver is located external to the body when in use.

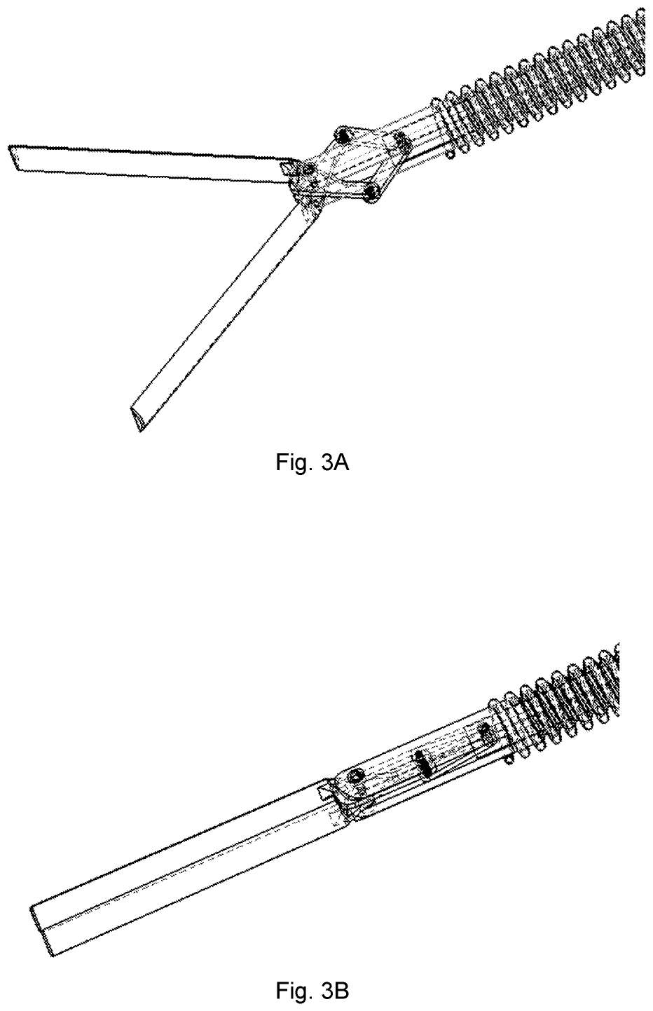

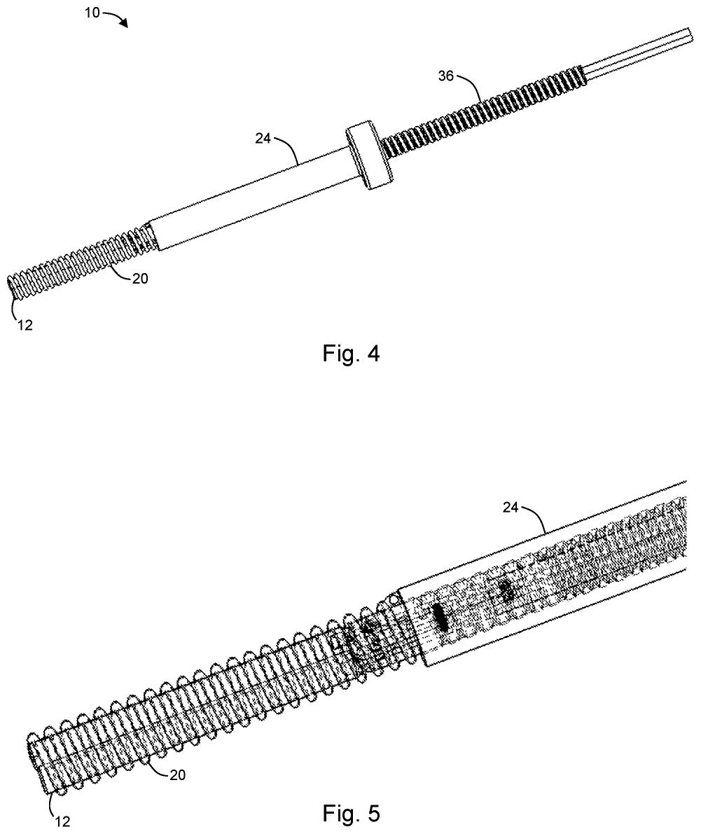

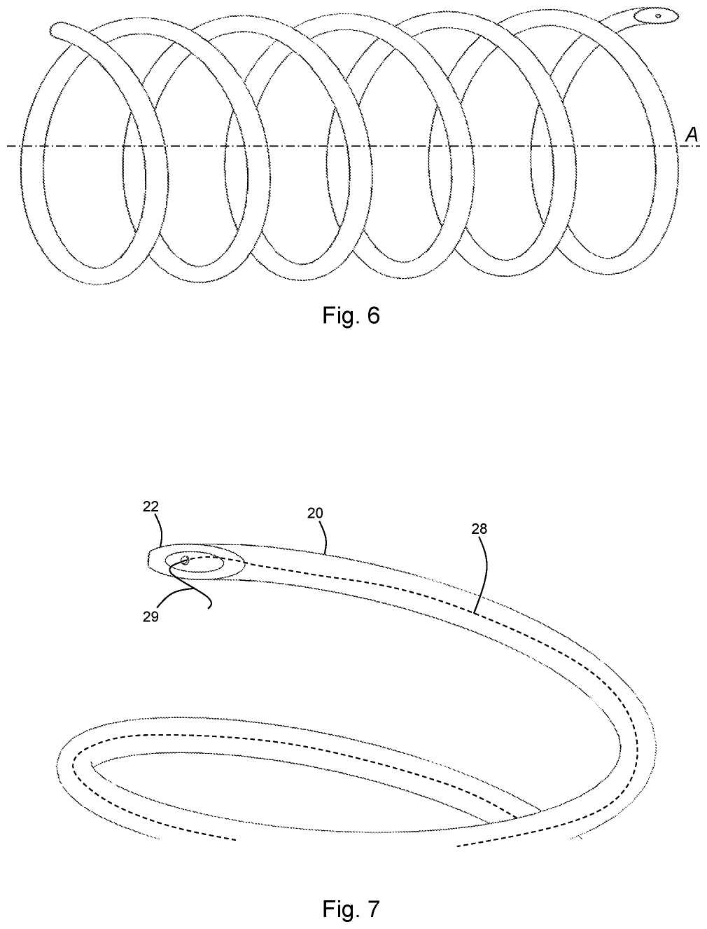

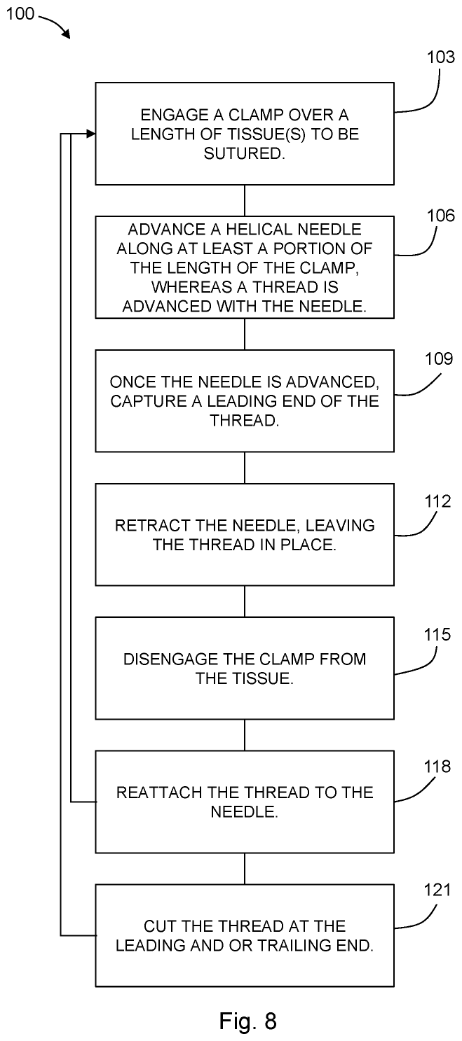

The helical needle 20 is configured such that advancement causes the needle to pierce material (e.g., one or more tissue(s)) contained within the clamp. The needle will pierce the material one or more times as it rotates along the length of the clamp. In some embodiments, the helical needle 20 may be disposed over the shaft 30 when in a withdrawn (retracted) position (FIGS. 3A and 3B). In this way, the clamp 12 may be opened (FIG. 3A) and closed (FIG. 3B) over the tissue(s). When the clamp 12 is closed (clamped) on the tissue(s) to be sutured, the helical needle 20 may be advanced along a length of the clamp 12 (see, e.g., FIGS. 4 and 5)—i.e., along the longitudinal length of the clamp 12. For example, the helical needle may be advanced over an exterior of the clamp. The shaft and/or the clamp may include a guide or guides (not shown) to guide the helical needle during advancement and retraction. The device may further include a sheath configured to enclose at least a portion of the helical needle when in a retracted position.

The needle 20 is configured such that the needle may be advanced over the clamp 12 when the joint 34 is straight (i.e., such that the clamp is coaxial with the longitudinal axis of the shaft 30) or when the joint 34 is bent (i.e., at an angle>0° from the longitudinal axis of the shaft 30). For example, the needle may be made from metal (e.g., surgical-grade steel), or rigid plastics or composites (e.g., carbon fiber). A diameter of the needle may also be configured for advancement over the bent joint. In a non-limiting, experimental embodiment, it has been found that a surgical steel needle with a diameter of 0.065″ was able to negotiate advancement over a bent joint of the shaft. In some embodiments, a tip 22 of the needle may be cut (i.e., sharpened) at an angle such that a point of the tip is at a radial exterior of the helix form (see FIG. 4). In experimental embodiments, the radial exterior needle tip has proven advantageous when used with a bent joint of the shaft.

The needle 20 may have a gauge of, for example, between 20 G and 10 G (Birmingham gauge; approximately 0.91-3.4 mm, outer diameter), inclusive, but may be smaller or larger as desired for the application. The overall diameter of the helix shape may be, for example, between 0.25-2.0 in (approximately 6.35-50.8 mm), inclusive, but may be smaller or larger as desired for a particular application. In a particular embodiment, a device is designed for use with a 15 mm port size. As such, the helix shape has a diameter which is less than 15 mm. Generally, an overall diameter of the device (diameter of the shaft, clamp, needle, driver, etc.) is less than a port size used in a particular procedure.

With reference to FIG. 7, the device 10 further includes a suturing thread 28 for suturing the tissue contained in the clamp 12. The suturing thread 28 has a leading end 29 which may be attached to the needle 20 (see, e.g., FIG. 4). In some embodiments, the leading end 29 is attached to (i.e., near) the tip 22 of the needle 20. In an example, the suturing thread 28 may be attached by way of a spring-loaded, multi-prong grasper as is known in the art. In other embodiments, the suturing thread 28 is attached by way of being tied to an eye of the needle 20. In other embodiments, the suturing thread is attached to the tip of the needle by nature of being disposed through a lumen of the needle. Other techniques for attachment including, for example, adhesives, will be apparent in light of the present disclosure. In some embodiments, the needle 20 is hollow and the suturing thread 28 is disposed through the hollow portion (lumen) of the needle 20 emerging at the tip 22. In other embodiments, the suturing thread may be generally external to the needle and attached at the tip. The needle 20 and suturing thread 28 are configured such that as the needle 20 is advanced along a length of the clamp—for example, through the guide 18 of the clamp 12—and through portions of the tissue(s) held by the clamp 12, the suturing thread 28 is carried through the tissue as well.

The number of sutures possible through the held tissue will generally match the number of helixes. For example, if a helix-shaped needle 20 includes 24 helixes, there will generally be a result of up to 24 sutures (assuming tissue was properly placed in the clamp 12, etc.) Note that the resulting suture is not 24 individual sutures, but a running suture or “continuous stitch”—i.e., a continuous suture having only the end stitches tied off. The needle 20 may have, for example, between 12 and 24 helixes, inclusive, but may have more helixes or fewer helixes. The needle 20 may have a length of, for example, between 3 inches and 6 inches (approximately 76.2-152.4 mm), inclusive, but may be longer or shorter.

The device 10 may include a trap 40 configured to engage with the tip 22 of the needle 20 when the needle 20 has been completely advanced through the clamp 12. By engage the needle 20, it is intended that the trap 40 is configured to capture the suturing thread 28—e.g., at least a portion of the thread, for example, at or near the leading end 29 of the suturing thread 28. In some embodiments, the trap 40 is configured such that the tip 22 of the needle 20 enters the trap 40. In some embodiments, the tip 22 is removable, and the trap 40 may be configured to remove and retain the tip 29 (along with at least a portion of the leading end 29 of the thread) within the trap 40. By capturing the suturing thread 28, the helical needle 20 may be retracted through the clamp 12 while leaving the suturing thread 28 in place. In other embodiments, the suturing thread may be held using another tool (e.g., laproscopic tool, etc.) while the needle is retracted.

In some embodiments, the once retracted, leaving the suture thread 28 in place, the thread 28 may be reattached to needle, such as, for example, at the tip 22 of the needle 20 (i.e., at a portion of the thread spaced apart from the in-place portion of the thread). The needle may include a feature, such as, for example, a hook, a slit, a clamp, or other etc. to which the thread may be attached. For example, the thread may be reattached by tying the thread to the needle (e.g., to a feature of the needle). Once the thread is reattached to the needle, the needle can be re-advanced through the tissue. For example, another portion of the tissue(s) can be clamped and the suture can continue from the earlier, in-place suture. In some embodiments, the suturing thread is disposed through the shaft and into an end of the needle engaged with the driver. In some embodiments, the suturing thread enters the needle at a location of the needle near the driver.

In some embodiments, the device 10 may further include one or more cutters to sever the suturing thread at the leading end and/or a trailing end. In this way, the device 10 may be removed from the body while leaving the suturing thread in place through the tissue(s).

In another aspect, the present disclosure may be embodied as a method 100 for automatic suturing. For example, the method 100 may be used to create a running suture over a length of tissue (or tissues). A clamp is engaged 103 over a length of tissue (or tissues) to be sutured. For example, where bowel may need to be sutured closed over a length of the bowel, the clamp is positioned over the length of the bowel such that the two tissue portions to be sutured together are within the clamp. The clamp may be engaged 103 to retain the tissue therein. The clamp may be engaged 103 using a lock configured to prevent the clamp from opening.

A helical needle is advanced 106 along at least a portion of a length of the clamp. For example, the needle may be advanced over the clamp with or without a guide or guides. The clamp and the helical needle may be of any configuration as defined herein. The helical needle is advanced 106 in a corkscrew-like fashion—i.e., advanced by rotating the needle about a central longitudinal axis of the helix shape. By advancing along at least a portion of the length of the clamp, the needle passes through the tissue(s) to be sutured. Based on the clamp and needle configuration and/or the manner of clamping the tissue(s) in the clamp, the needle may pass through the tissue(s) once with each rotation of the needle or twice with each rotation of the needle. The needle may be advanced by an actuator which is controlled by a surgeon. For example, each time the actuator is triggered (e.g., squeezing a trigger, depressing a button, toggling a switch, depressing a foot pedal, etc.), the needle may advance by one rotation and may puncture the tissue(s) one or two times.

In some embodiments, the needle is a hollow needle and includes a thread (a suturing thread) disposed at least partially through the hollow interior of the needle. In this way, the suturing thread is carried through the clamp and tissue by the helical needle. In some embodiments, the suturing thread is outside of the needle and attached to a tip portion of the needle. In this way, the suturing thread is pulled through the tissue by needle as the needle is advanced 106 through the clamp. In some embodiments, the needle is advanced 106 one rotation at a time (i.e., with each actuation of the device). In some embodiments, actuating the device may advance the needle by less than one rotation. For example, the actuator advances the needle by half of a rotation, a third of a rotation, a quarter of a rotation, or any other rotation less than a full rotation. As such, the tissue may be punctured by the needle one or two times for each actuation of the device or not punctured at all by actuation. In other embodiments, the needle is advanced 106 by more than one rotation at a time. For example, a surgeon may cause the needle to advance, and such advancement may continue until the needle has travelled through its entire path through the clamp. In some embodiments, the advancement 106 of the needle may occur only while activated by an operator. For example, a surgeon may squeeze a trigger to cause the needle to advance and the needle may continue to advance until the surgeon releases the trigger. The needle may advance at a selectively variable rate. For example, the advancement rate may be proportional to a position of an actuator (e.g., variable trigger, foot pedal position, etc.) In another example, the advancement rate is determined according to a control that is separate from the actuator such as, for example, a multi-setting switch, dial, rotatable knob, etc. Other advancement schemes will be apparent in light of the present disclosure.

A leading end of the suturing thread may be captured 109 or otherwise fixed at a location of the tissue once the needle is advanced 106 along the clamp. For example, the suturing thread may be captured when the tip of the helical needle is advanced to an end position. The end location may be at a distal end of the clamp. In some embodiments, the end location may be at a position spaced apart from the distal end of the clamp. In some embodiments, the end location may be selected by a user according to the application. For example, in some embodiments as further described below, a trap is positioned on the clamp to capture 109 at least a portion of the suturing thread. In some embodiments, the tip of the needle is removable, and the tip is captured 109 along with the at least a portion of the suturing thread. In other embodiments, the suturing thread is captured manually by an operator (for example, a surgeon) using a separate device.

Once the suturing thread is captured 109, the needle is retracted 112 back along the length clamp. For example, the needle is retracted 112 using an opposite corkscrew movement from the advancing movement—i.e., by oppositely-rotating the needle about the central longitudinal axis of the helix shape. Because at least a portion of the suturing thread is captured 109, the suturing thread is left in place during retraction 112 of the needle. By left in place, it is intended that the suturing thread remain disposed through the tissue(s) at the locations where the needle passed through the tissue(s). In embodiments where the suturing thread is disposed through a hollow portion of the needle, the needle is retracted 112 by passing over the suturing thread (the suturing thread slides through the hollow portion, thereby remaining in place once the needle is retracted).

The clamp is disengaged 115 from the tissue. In some embodiments, the thread may be reattached 118 to the needle, for example, to a tip of the needle. In this way, the device may be immediately used again to perform another suture on the same or a different tissue. By immediately, it is intended that a subsequent suture may be performed without the need to remove the device from the body of the individual. In some embodiments, the suturing thread may be cut 121 at the leading end and/or a trailing end such that the clamp and needle may be moved or removed while leaving the suturing thread in place.

The leading end of the suturing thread and/or the trailing end of the suturing thread may be fixed to complete the suture. In some embodiments, one or both of the ends are fixed 121 by knotting the thread (securing the thread to itself). In some embodiments, the suturing thread is knotted such that one or both ends cannot pull through the tissue. In some embodiments, the suturing thread is configured to swell to a thicker diameter (in this case, thicker means a diameter greater than an initial diameter of the suturing thread) thereby retaining the suturing thread in the tissue. Such threads are known in the art. Before fixation of one or both ends, the suturing thread may be adjusted such that the suture is tightened as desired. One or both ends of the suturing thread may be fixed before or after disengagement 115 of the clamp.

In some embodiments, the helical needle is advanced 106 and/or retracted 112 using an electric motor. In some embodiments, the helical needle is advanced 106 and/or retracted 112 using by way of a manual operation (e.g., handle, hand crank, etc.)

It has been found that, in some cases, a thread diameter may advantageously be sufficiently smaller than an inside diameter of the needle in order be more easily moved through the needle. For example, when using a 16RW hypodermic tube (having an inside diameter of 0.047 inches (1.19 mm) and an outside diameter of 0.065 inches (1.65 mm)) as the needle, thread sizes of 4-0 (0.15 mm diameter) or smaller provide satisfactory performance. In a particular embodiment, a 4-0 poly suture was successfully used with a helical needle formed from 16RW stainless steel hypodermic tubing. Other ratios of suture diameter to needle insider diameter may be acceptable and may depend on one or more of the suture material, the needle material, the needle helix parameters (helix diameter, helix angle), etc. Acceptable ratios of suture diameter:needle inside diameter may range from 1:6 to 1:10, inclusive, and all values therebetween. In some embodiments, the ratio may be even higher or lower than these exemplary values/ranges. In some embodiments, the ratio of the suturing thread diameter to the helical needle inside diameter is greater than 1:8 (e.g., 1.81, 1.82, . . . , 1:9, 1.91, . . . , 1:10, etc.)

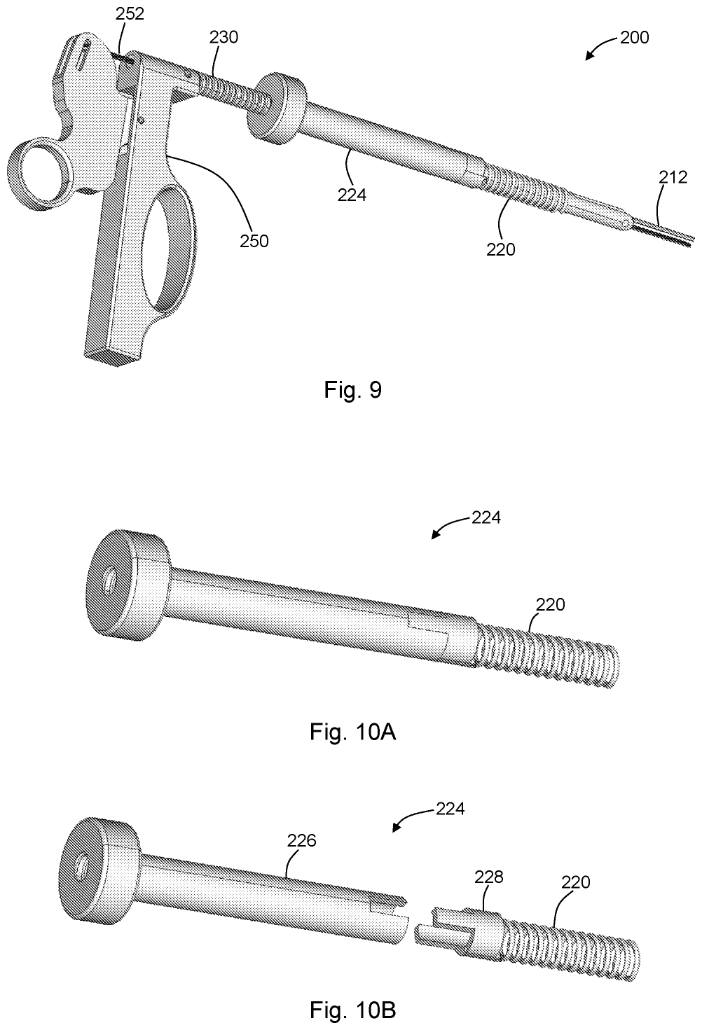

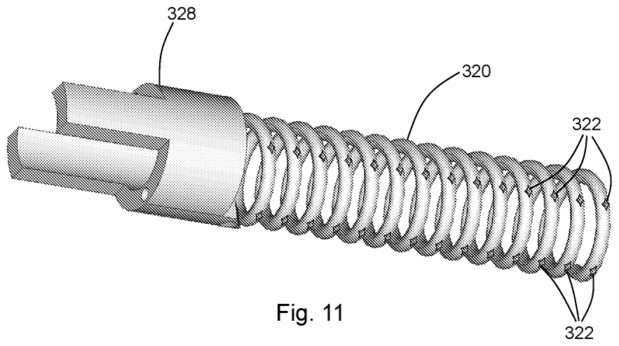

FIG. 9 depicts another embodiment of a device 200 for automatic suturing. The device 200 includes a shaft 230 with a screw thread for advancing a driver 224 when the driver 224 is rotated. The driver 224 is configured to drive a needle 220 as described above. A clamp 212 is operable to secure tissue for suturing when the needle 220 is advanced over the clamp 212. The device 200 also includes a handle 250 which is configured to operate the clamp 212 using a rod 252. FIGS. 10A and 10B show a detail view of the driver 224 and needle 220. The embodiment of device 200 utilizes a two-piece driver 224 having a driver body 226 and a driver head 228. The driver body 226 is configured to rotate the driver head 228, but the design allows the driver body to advance at a rate that is different than the driver head. In this way, any differences between the helix configuration and the screw configuration (of the shaft) can be tolerated.

FIG. 11 depicts an embodiment of a driver head 328 and needle 320. The needle of this embodiment includes a plurality of windows 322—orifices through the needle wall, allowing external access to the lumen of the needle. In this way, a suture thread may be loaded into the needle by sequentially inserting the thread from a window 322 to the next window 322, and so on until the needle is loaded with suture thread. In the embodiment shown in FIG. 11, the needle includes a window 322 every 90° of the helix (i.e., four windows per rotation of the helix). Other embodiments utilize fewer windows or more windows.

### FURTHER EXAMPLES

Example 1. A device for automatic suturing, comprising: a shaft having a longitudinal axis, the shaft having a joint rotatable about a joint axis which is at an angle>0° to the longitudinal axis; a clamp at a distal end of the shaft and configured to be positioned over a length of tissue to be sutured; a driver configured to engage with a screw of the shaft so as to translate along a length of the shaft when rotated; a helical needle connected to the driver such that the helical needle is rotated while advancing or retracting over a length of the clamp and over the joint of the shaft.

Example 2. The device of example 1, wherein a tip of the helical needle is at a radially exterior portion of the helical needle for movement over the joint of the shaft.

Example 3. The device of any one of examples 1 or 2, further comprising a suturing thread having a leading end near a tip of the helical needle.

Example 4. The device of example 3, wherein the helical needle is hollow and the suturing thread is disposed through the helical needle.

Example 5. The device of any one of examples 3 or 4, further comprising at least one cutter for severing the suturing thread.

Example 6. The device of any one of examples 3 or 4, wherein the suturing thread has a diameter, the helical needle has an inside diameter, and the ratio of the suturing thread diameter to the helical needle inside diameter is in the range of 1:6-1:10, inclusive.

Example 7. The device of example 6, wherein the ratio of the suturing thread diameter to the helical needle inside diameter is greater than 1:8.

Example 8. The device of any one of examples 1-7, further comprising a lock for reversibly locking the clamp in a closed position.

Example 9. The device of any one of examples 1-8, wherein the helical needle is configured to be advanced over an exterior of the clamp.

Example 10. The device of any one of examples 1-9, further comprising a shaft attached to the clamp, and wherein the helical needle is configured to be disposed over the shaft when in a retracted position.

Example 11. The device of any one of examples 1-10, further comprising a motor operably connected to the driver.

Example 12. The device of any one of examples 1-11, wherein the helical needle further comprises one or more windows.

Example 13. A method for automatic suturing, comprising: engaging a clamp of a device according to any one of examples 1-12 over a first portion of tissue to be sutured; advancing the helical needle of the device along a length of the clamp such that the helical needle passes through the tissue to be sutured, and wherein a leading end of a suturing thread is attached near a tip of the helical needle; capturing the leading end of the suturing thread; retracting the helical needle back through the tissue so as to leave the suturing thread in place; and disengaging the clamp from the tissue.

Example 14. The method of example 13, further comprising fixing the leading end of the suturing thread and/or a trailing end of the suturing thread to form one or more sutures.

Example 15. The method of example 14, wherein the leading end of the suturing thread and/or the trailing end of the suturing thread are fixed by knotting.

Example 16. The method of any one of examples 13-15, wherein the clamp is locked in place over the tissue.

Example 17. The method of any one of examples 13-16, wherein the helical needle is advanced by a motor.

Example 18. The method of any one of examples 13-17, wherein the helical needle is advanced in single rotation increments.

Although the present disclosure has been described with respect to one or more particular embodiments, it will be understood that other embodiments of the present disclosure may be made without departing from the spirit and scope of the present disclosure.

1. A device for automatic suturing, comprising:
a shaft having a longitudinal axis, the shaft having a joint rotatable about a joint axis which is at an angle>0° to the longitudinal axis;
a clamp at a distal end of the shaft and configured to be positioned over a length of tissue to be sutured;
a driver configured to engage with a screw of the shaft so as to translate along a length of the shaft when rotated;
a helical needle connected to the driver such that the helical needle is rotated while advancing or retracting over a length of the clamp and over the joint of the shaft.

2. The device of claim 1, wherein a tip of the helical needle is at a radially exterior portion of the helical needle for movement over the joint of the shaft.

3. The device of claim 1, further comprising a suturing thread having a leading end near a tip of the helical needle.

4. The device of claim 3, wherein the helical needle is hollow and the suturing thread is disposed through the helical needle.

5. The device of claim 3, further comprising at least one cutter for severing the suturing thread.

6. The device of claim 3, wherein the suturing thread has a diameter, the helical needle has an inside diameter, and the ratio of the suturing thread diameter to the helical needle inside diameter is in the range of 1:6-1:10, inclusive.

7. The device of claim 6, wherein the ratio of the suturing thread diameter to the helical needle inside diameter is greater than 1:8.

8. The device of claim 1, further comprising a lock for reversibly locking the clamp in a closed position.

9. The device of claim 1, wherein the helical needle is configured to be advanced over an exterior of the clamp.

10. The device of claim 1, further comprising a shaft attached to the clamp, and wherein the helical needle is configured to be disposed over the shaft when in a retracted position.

11. The device of claim 1, further comprising a motor operably connected to the driver.

12. The device of claim 1, wherein the helical needle further comprises one or more windows.

13. A method for automatic suturing, comprising:
engaging a clamp of a device according to claim 1 over a first portion of tissue to be sutured;
advancing the helical needle of the device along a length of the clamp such that the helical needle passes through the tissue to be sutured, and wherein a leading end of a suturing thread is attached near a tip of the helical needle;
capturing the leading end of the suturing thread;
retracting the helical needle back through the tissue so as to leave the suturing thread in place; and
disengaging the clamp from the tissue.

14. The method of claim 13, further comprising fixing the leading end of the suturing thread and/or a trailing end of the suturing thread to form one or more sutures.

15. The method of claim 14, wherein the leading end of the suturing thread and/or the trailing end of the suturing thread are fixed by knotting.

16. The method of claim 13, wherein the clamp is locked in place over the tissue.

17. The method of claim 13, wherein the helical needle is advanced by a motor.

18. The method of claim 13, wherein the helical needle is advanced in single rotation increments.
